(19) United States
(12) Patent Application Publication
(10) Pub. No.: US 2026/0264064 A1
(43) Pub. Date: Sep. 10, 2026

(54) CATALYST AND PROCESS FOR PRODUCING METHANOL

(71) Applicant: Technische Universität Wien, Wien (AT)

(72) Inventors: Karin Föttinger, Wien (AT); Gernot Pacholik, Wien (AT)

(21) Appl. No.: 18/875,567

(22) PCT Filed: Jun. 20, 2023
(86) PCT No.: PCT/AT2023/060192

§ 371 Date: Dec. 16, 2024

(30) Foreign Application Priority Data
Jun. 20, 2022 (AT) A 50438/2022

(51) Int. Cl. B01J 27/051; B01J 23/06; B01J 35/30; B01J 37/02; B01J 37/04; C07C 29/153

(52) U.S. Cl. CPC B01J 27/051; B01J 23/06; B01J 35/394; B01J 37/0221; B01J 37/0236; B01J 37/04; C07C 29/153

(57) ABSTRACT

Catalyst, comprising a carrier and a catalytically active coating, wherein the catalytically active coating comprises a metal sulfide, wherein the carrier is zinc(II) oxide, wherein the content of metal sulfide is between 15 and 30 wt. %, preferably between 15 und 25 wt. %, particularly preferred between 15 and 20 wt. %, based on zinc(II) oxide.

The present invention relates to a method for catalytically producing methanol from carbon dioxide and hydrogen, wherein carbon dioxide is reacted with hydrogen over a catalyst consisting of zinc oxide and metal sulfide. Furthermore, the invention relates to a catalyst for producing methanol from carbon dioxide and hydrogen. Finally, the invention relates to the use of a catalyst for producing methanol from carbon dioxide and hydrogen.

### BACKGROUND OF THE INVENTION

Methanol is not only an important raw material for further processing to more complex chemical products, it also is an energy carrier for direct combustion or conversion in fuel cells. Synthetic methods for obtaining methanol often yield mixtures of methanol and other alcohols, which is why selective methods for obtaining methanol are of great importance.

Industrial methods for producing methanol according to the state of the art mainly work via hydrogenation of carbon monoxide or carbon dioxide, in each case under high pressures, on suitable catalysts. Hydrogenation starting from synthetic gas leads to both reactions, however, the yield of CO₂ hydrogenation is still in need of improvement.

Liu et al., Journal of the Taiwan Institute of Chemical Engineers, 76 (2017), page 18, describe the production of higher alcohols via CO₂ hydrogenation with a Mo—Co—K-sulfide catalyst, wherein the catalyst may, e.g., have MoS₂.

Qi et al., Catalysis Communication, 4 (2003), page 339, describe CO hydrogenation by means of K/MoS₂. They also describe the addition of manganese to the catalyst, and finally they describe a Ni/Mn/K/MoS₂ catalyst to be suitable for the hydrogenation of CO. The result shows very high selectivity for alcohols with a total yield of 81.7%. Thereof, 45.8% are methanol and 53.3% are higher alcohols (Cₙ alcohols with n=2, 3, 4 and 5).

Zeng et al., Applied Catalysis B: Environmental, 246 (2019), page 232, describe the production of higher alcohols with at least three carbon atoms (Cₙ alcohols with n≥3) by means of CO hydrogenation over MoS₂ promoted with potassium.

Ramadan A. Geioushy, Islam M. Hegazy, Said M. El-Sheikh, Osama A. Fouad; “Construction of 2D MoS2@ZnO heterojunction as superior photocatalyst for highly efficient and selective CO2 conversion into liquid fuel” Journal of Environmental Chemical Engineering, Volume 10, Issue 2, 2022, 107337 (https://doi.org/10.1016/j.jece.2022.107337) describe a catalyst consisting of a 1:1 mixture of MoS₂ and ZnO. The catalyst is used in a photochemical method for converting CO₂ into Methanol.

### SUMMARY OF THE INVENTION

Selective methods for producing methanol based on CO₂ known from the state of the art require expensive catalysts and complex process conditions and provide low yields. The catalyst described in Geioushy et al. is much better than other catalysts with regard to specificity, however, process execution in Geioushy et al. is laborious due to the necessity of UV irradiation and aqueous process conditions.

It is the object of the present invention to provide a selective and cost-efficient method as well as a catalyst for a simple, selective hydrogenation of CO₂ to methanol with high selectivity. In addition, the catalyst should be sulfur-tolerant, i.e. tolerant against traces of sulfur compounds, which are, e.g., present in flue gas, which may be an educt for the synthesis of methanol.

This object is achieved by a catalyst comprising a carrier and a catalytically active coating, wherein the catalytically active coating comprises a metal sulfide, wherein the carrier is zinc (II) oxide, wherein the content of metal sulfide is between 15 and 30 wt. %, preferably between 15 and 25 wt. %, especially preferred between 15 and 20 wt. %, based on zinc (II) oxide.

The invention is based on the finding that catalysts comprising a carrier of ZnO and a catalytically active coating with a metal sulfide catalyze CO₂ hydrogenation with very high selectivity and high yields. Here, it is decisive that the catalyst comprises not only a mixture of ZnO and metal sulfide, but that ZnO acts as a carrier. Also, the ratio of ZnO to metal sulfide is essential because higher amounts of metal sulfide have a negative impact on the reaction conditions. With the correct structure of carrier and catalytically active coating as well as the correct quantity ratio of ZnO and metal sulfide, the reaction of CO₂ and H₂ can be performed without UV irradiation and in a gas phase with very quick reaction kinetics.

The catalytic property of metal sulfides during production is basically known, usually with regard to long-chain alcohols. However, the inventors have now found out that the carrier plays an important role for the selectivity of the catalyst, and that only ZnO leads to a selective formation of methanol. Other carriers show this selectivity to a much lesser extent.

Almost no higher alcohols are formed in this method, and in addition, the amounts of byproducts like CO or CH₄ that are formed are low. However, the inventors have now found out that the carrier plays a special role for the selectivity of the catalyst, with ZnO resulting in a much more selective formation of methanol. Other carriers do not show this distinctness.

It has been shown that molybdenum (IV) sulfide (MoS₂) is particularly suitable as metal sulfide.

MoS₂ is present as a coarse layer on the ZnO carrier. Its particle size is in a range of 100 nm to 1 μm. In the finished catalyst, Zn is present in the oxidation state +II and as a hexagonal ZnO phase.

The invention also relates to a selective method for producing methanol (CH₃OH, MeOH) from carbon dioxide (CO₂) and hydrogen (H₂), wherein CO₂ is reacted with H₂ in the gas phase over a catalyst, wherein the catalyst has a carrier and a catalytically active coating, wherein the catalytically active coating comprises a metal sulfide, wherein the carrier is zinc (II) oxide (ZnO), wherein the content of metal sulfide is between 15 und 30 wt. %, preferably between 15 und 25 wt. %, especially preferred between 15 and 20 wt. %, based on zinc(II) oxide.

The exact reaction mechanism is still unknown, however, the two-step reaction sequence preceded by a RWGS step (Reverse Water-Gas Shift Reaction) and subsequent CO hydrogenation to methanol is unlikely because the catalyst hardly showed any conversion of CO with H₂ to CH₃OH. Consequently, the high yield and selectivity are all the more surprising.

The invention thus also relates to the use of a catalyst, wherein the catalyst has a carrier and a catalytically active coating, wherein the catalytically active coating comprises a metal sulfide, wherein the carrier is zinc(II) oxide (ZnO), for producing methanol from CO₂ and H₂.

Below, further advantageous details of the catalyst and the method are explained in more detail, wherein both aspects of the invention are coordinated with each other.

The metal sulfide of molybdenum (IV) sulfide (MoS₂) has proven to be a particularly well suited catalytically active material

Increased pressure compared to standard conditions has proven advantageous as reaction condition during the method. Consequently, it is preferably provided that the reaction takes place at a pressure ≥10 bar. For example, the pressure can be 10 bar to 200 bar or 10 bar to 100 bar. In one embodiment variant, the pressure was between 18 bar and 23 bar.

Basically, the reaction can take place within a wide temperature range. Suitable temperatures are, e.g., between 160° C. and 260° C., preferably between 180 und 220° C.

Preferably it is provided that the partial pressure of CO₂ to H₂ is approximately 1 to 2.5 to 3.5, preferably approximately 3. This means that the partial pressure of hydrogen should be 2.5 to 3.5 times higher than the partial pressure of CO₂.

In one embodiment variant, the CO₂ can originate from flue gas. In this case, the inventive method is a selective method for producing methanol from CO₂ and H₂, wherein the CO₂ source is flue gas, wherein CO₂ is reacted with H₂ over a catalyst, wherein the catalyst has a carrier and a catalytically active coating, wherein the catalytically active coating comprises a metal sulfide, wherein the carrier is zinc(II) oxide. Such a method is suited for recycling flue gas.

Even though metal sulfides are considered as catalytically active materials, MoS₂ is particularly efficient.

In one aspect, the invention relates to a method for producing a catalyst, wherein the catalyst has a carrier and a catalytically active coating, wherein the catalytically active coating comprises a metal sulfide, wherein the carrier is zinc (II) oxide (ZnO), comprising the steps:
(i) forming a mixture of water, ammonium thiomolybdate, in particular (NH₄)₂MoS₄ or a hydrate of (NH₄)₂MoS₄, and ZnO;
(ii) stirring the mixture; and
(iii) then evaporating the water from the mixture.

The quantity ratios (wt.) of ammonium thiomolybdate, in particular (NH₄)₂MoS₄ or a hydrate of (NH₄)₂MoS₄, to ZnO are preferably 54 to 163 (NH₄)₂MoS₄) to 1 (ZnO). The amount of water is 200 to 1000 (Gew.) based on the ZnO (1) used. It is therefore preferably provided that the ratio of ammonium thiomolybdate, (NH₄)₂MoS₄ or a hydrate of (NH₄)₂MoS₄, to ZnO is between 54 to 163 wt. %.

Preferably, step (iii) is performed at temperatures below 100° C. in order to prevent a reaction with oxygen in the air. A temperature range of 60° C. to 80° C. is advantageous.

Therefore, it is beneficial if step (iii) is performed at a pressure below 1 bar. A pressure below 500 mbar, especially preferred below 250 mbar, is advantageous.

### DETAILED DESCRIPTION OF THE INVENTION

Further advantages and details of the invention are shown in the accompanying figures and explained in more detail in the following description.

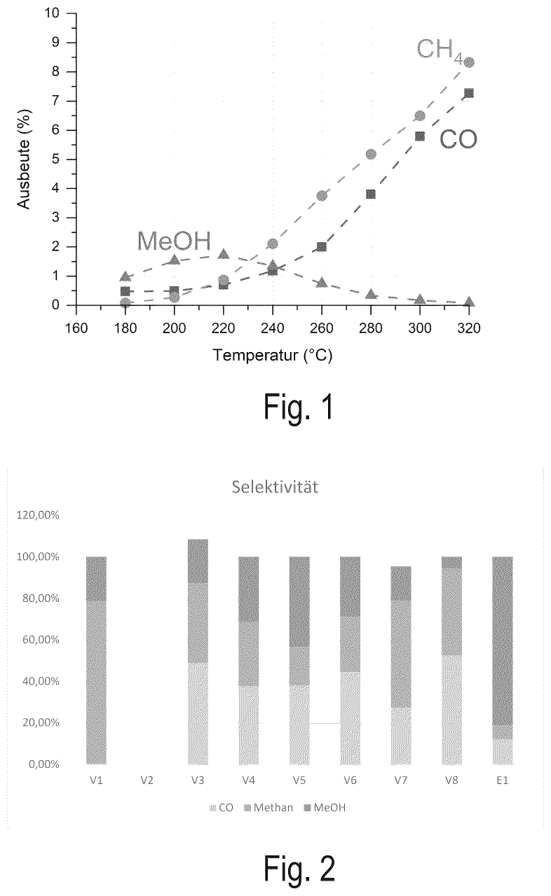

FIG. 1 shows the reaction yield of methanol, CH₄, and CO of the reaction of CO₂ with H₂ over an inventive catalyst as a function of the temperature in an inventive method.

FIG. 2 shows a comparison of the yields of reactions of CO₂ with H₂ over an inventive catalyst compared to seven non-inventive catalysts.

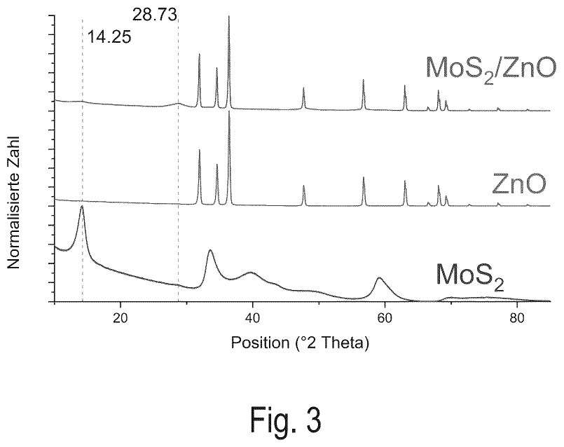

FIG. 3 shows x-ray diffraction patterns (XRD) of an inventive catalyst with MoS₂ as catalytically active layer and ZnO as carrier compared to ZnO without catalytic layer and MoS₂ without carrier.

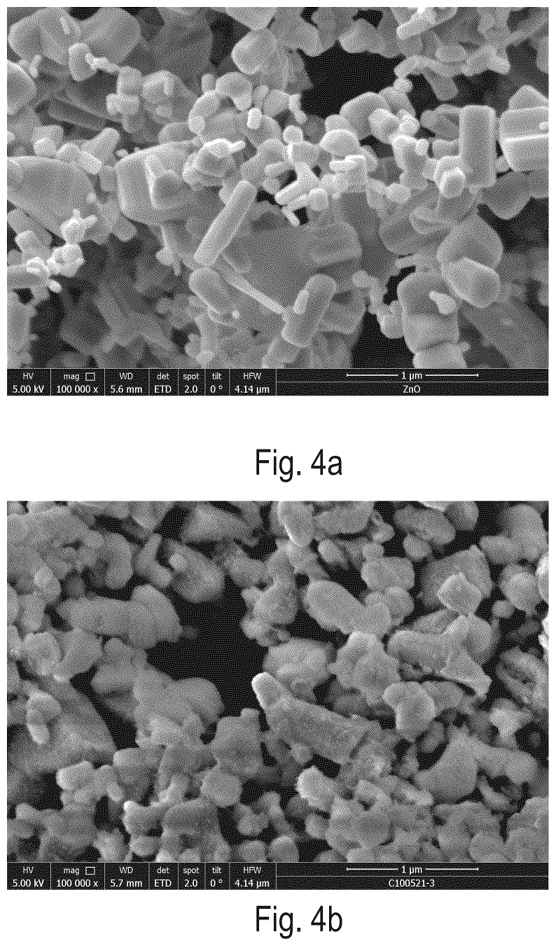

FIG. 4a shows scanning electron microscope images of ZnO.

FIG. 4b shows scanning electron microscope images of an inventive catalyst with MoS₂ as catalytically active layer with ZnO.

FIG. 1 shows the reaction yields of methanol, CH₄, and CO as a function of the temperature in an inventive method. Here, one gram of an inventive catalyst (consisting of a carrier of ZnO with a catalytically active coating of 16.7 wt. % MoS₂) is reacted with CO₂ and H₂. The catalyst was first pre-treated at 400° C. and 100% H₂ for 4 h at a pressure of 21 bar and a total flow of 5 mlN.

Explanation: The total flow at the gas mixture as conducted over the catalyst is:

$$\frac{5 mlN}{gkat * min}$$

In this formula, “mlN” represents milliliter under normal or standard conditions, i.e. at 273.15 K or 0° C. and 1 bar of pressure. A normalization to normal conditions is performed because 1 ml would have a higher amount of substances under 21 bar than under 1 bar; therefore, the flow is converted and referenced to the volume flow under normal conditions.

The reaction itself was examined starting at 180° C. in 20° C. steps, wherein a gas mixture of CO₂ with H₂ and He were applied at 21 bar. The partial pressure of CO₂ was 20%, the partial pressure of H₂ was 60%, and the partial pressure of He was 20%.

Is can be clearly seen that the maximum yield of methanol is achieved at approximately 220° C., while only few byproducts are formed at this temperature. At a higher temperature, the formation of methane (CH₄) increases, while the yield of methanol decreases. In addition, the amount of carbon monoxide (CO) formed increases with a higher temperature. The ideal temperature range is approximately 180° C. to 230° C.

FIG. 2 shows a comparison of eight different catalysts (V1 to V8) and the inventive catalyst. The compositions of the individual catalysts are shown in Table 1.

TABLE 1
Compositions of the catalysts for the
examples according to FIG. 2.
V1 to V8 are comparative examples, E1 is inventive:

| | | | | Amount of |
|---|---|---|---|---|
| | | | | catalytically |
| | | | Amount | active metal |
| | | | of | coating based |
| | | Catalytically | metal | on total |
| | | active | sulfide | mass |
| Figure | Carrier | coating | (wt. %) | (wt. %) |
| V1 | AlO(OH) | MoS₂ | 16.7 | 10 |
| V2 | MnO₂ | MoS₂ | 16.7 | 10 |
| V3 | C* | MoS₂ | 16.7 | 10 |
| V4 | AlO(OH) | MoS₂ | 3.34 | 2 |
| V5 | MgAlOₓ | MoS₂ | 16.7 | 10 |
| | (Mg:Al = | | | |
| | 2:1) | | | |
| V6 | MgAlOₓ | MoS₂ | 16.7 | 10 |
| | (Mg:Al = | | | |
| | 1:2) | | | |
| V7 | ZrO₂ | MoS₂ | 16.7 | 10 |
| V8 | CeO₂ | MoS₂ | 16.7 | 10 |
| E1 | ZnO | MoS₂ | 16.7 | 10 |

*Activated charcoal

The reaction conditions of the examples shown in FIG. 2 at the beginning of the reaction are always identical. The reaction yields of methanol, CH₄, and CO are shown in FIG. 2. In each case, one gram of catalyst (catalytically active coating always MoS₂) was reacted with CO₂ and H₂. The catalyst was first pretreated at 400° C. and 100% H₂ for 4 hours at a pressure of 21 bar and a total flow of 5 mlN.

The reaction itself was examined at 180° C., wherein a gas mixture of CO₂ with H₂ and He was applied at 21 bar. The partial pressure of CO₂ was 20%, the partial pressure of H₂ was 60%, and the partial pressure of He was 20%. While the catalytically active coating was always MoS₂, there were different distributions of reaction products depending on the carrier material. The highest specificity for methanol was seen with ZnO as carrier material, while the other materials provided high amounts of CH₄ and/or CO.

FIG. 3 shows x-ray diffraction patterns (XRD) of an inventive catalysts with MoS₂ as catalytically active layer and ZnO as carrier compared to ZnO without catalytic layer and MoS₂ without carrier. The XRD for MoS₂/ZnO shows that the ZnO structure is adopted. It is assumed that there are 3-dimensional MoS₂ islands with low crystallinity on the ZnO carrier.

FIG. 4a and FIG. 4b show scanning electron microscope images of ZnO and MoS₂ with ZnO as carrier. In the image of FIG. 4b it can be seen that the ZnO particles are covered by MoS₂.

1. A catalyst, comprising a carrier and a catalytically active coating, wherein the catalytically active coating comprises a metal sulfide, wherein the carrier is zinc (II) oxide, wherein the content of metal sulfide is between 15 and 30 wt. % based on zinc (II) oxide.

2. The catalyst according to claim 1, wherein the metal sulfide comprises molybdenum (IV) sulfide (MoS₂).

3. A method for producing methanol (CH₃OH) from carbon dioxide (CO₂) and hydrogen (H₂), wherein CO₂ is reacted with H₂ in the gas phase over a catalyst according to claim 1.

4. The method according to claim 3, wherein during the reaction, a pressure of ≥10 bar is set.

5. The method according to claim 3, wherein during the reaction, a temperature of between 160° C. and 260° C.

6. The method according to claim 3, wherein the partial pressure ratio of CO₂ to H₂ is approximately 1 to 2.5 to 3.5.

7. The method according to claim 3, wherein the reaction is performed in the absence of UV light.

8. The method according to claim 3, wherein the CO₂ originates from flue gas.

9. A method for producing a catalyst according to claim 1, wherein the catalyst has a carrier and a catalytically active coating, wherein the catalytically active coating comprises a metal sulfide, wherein the carrier is zinc (II) oxide (ZnO), the method comprising the steps of:
(i) forming a mixture of water, ammonium thiomolybdate, in particular (NH₄)₂MoS₄ or a hydrate of (NH₄)₂MoS₄, and ZnO;
(ii) stirring the mixture; and
(iii) then evaporating the water from the mixture.

10. The method according to claim 9, wherein step (iii) is performed at a pressure below 1 bar.

11. A catalyst according to claim 1, wherein the content of metal sulfide is between 15 und 25 wt. % based on zinc (II) oxide.

12. A catalyst according to claim 1, wherein the content of metal sulfide is between 15 and 20 wt. % based on zinc (II) oxide.

13. The method according to claim 3, wherein during the reaction a pressure between 18 bar und 23 bar is set.

14. The method according to claim 3, wherein during the reaction a temperature of between 180° C. to 220° C. is set.

15. The method according to claim 3, wherein the partial pressure ratio of CO₂ to H₂ is approximately 3.

16. The method according to claim 9, wherein evaporating the water from the mixture in step (iii) of is performed below 100° C.

17. The method according to claim 9, wherein evaporating the water from the mixture in step (iii) of is performed at 60° C. to 80° C.

18. The method according to claim 9, wherein step (iii) is performed at a pressure below 500 mbar.
